(19) United States
(12) Patent Application Publication
(10) Pub. No.: US 2013/0131587 A1
(43) Pub. Date: May 23, 2013

(54) MEDICAL EQUIPMENT SYSTEM

(71) Applicants: Holger Jungmann, Gelsenkirchen (DE); Michael Schietzel, Herdecke (DE)

(72) Inventors: Holger Jungmann, Gelsenkirchen (DE); Michael Schietzel, Herdecke (DE)

(21) Appl. No.: 13/639,419

(22) PCT Filed: Apr. 11, 2011
(86) PCT No.: PCT/EP2011/001792

§ 371 Date: Jan. 22, 2013

(30) Foreign Application Priority Data
Apr. 12, 2010 (DE) 10 2010 014 703.6

(51) Int. Cl. A61M 5/172

(52) U.S. Cl. CPC A61M 5/1723
USPC 604/66

(57) ABSTRACT

The invention is directed to a medical equipment system by means of which the provision of medication to a patient is adapted according to continuously recorded physiological measurement signals. The object of the invention is to find ways by which medication can be administered to a patient with a high degree of accuracy. This object is achieved, according to the invention, by a medical equipment system with a spectrometer for recording measurement signals from vital tissue of a patient, in particular for continuously determining the material composition of body liquids and also of tissue-bound substances possibly present only temporarily in the corresponding tissue area, with a signal processor for processing the measurement signals supplied from the spectrometer and for generating a medication signal, and with medication equipment for administering a medicament according to the medication signal.

The invention concerns a medical equipment system for coordinating, in accordance with continuously detected physiological measuring signals, a medication of a patient.

The object of the invention is to create solutions by which a medication of a patient can be reached with high precision.

This task is solved according to the invention by a medical equipment system with:

a spectrometer device for collecting measuring signals from living tissue of a patient, especially for the continuous determination of the composition of body liquids as well as of vascular-bound substances that may be only temporarily present in the corresponding tissue area,

a signal processing device for processing of the is measuring signal delivered by the spectrometer device and for generating a medication signal; and

a medication device for administering a medicament in accordance with the medication signal, wherein the signal processing device presents a first interface for the connection of the spectrometer device, a second interface device for the connection of the medication device, and a third interface device in which over the third interface arrangement the evaluation of the measuring signal delivered by the spectrometer device is configurable as well as the determination procedure for the medication signal.

It is thus advantageously possible to create a medication system suitable for everyday treatment in which a medication adapted for the patient can be adjusted on the basis of continuous spectrometric measurements. The signal processing device can be controlled and configured advantageously after the connection of the system to the patients. By the third interface device preferably also the raw data generated by the spectrometer device can be read.

Preferably the spectrometer device and the signal processing device are configured in such a way that by these media the detection of the spectra is done with such a high sampling rate that in this way cell dynamic processes, especially vasomotion effects, can be recognized.

In this way it is possible to adjust the medication taking into is account conditions that can be recognized by closely correlated cell dynamic phenomena.

According to a particular aspect of the invention preferably by the third interface device signal processing programs can be loaded into the signal processing device, which are kept ready at first in an operator computer system, for example as so-called tools.

The configuration of the signal processing device is done preferably by a user surface, which makes it possible to determine the function of the signal processing device by Drag and Drop routines, as well as by graphically supported range and parameter selections.

By the Drag and Drop function for example the substances and phenomena which are relevant in terms of control can be selected.

By the range selection the importance of these selected substances and phenomena can be adapted. Preferably for typical medication necessities special control concepts are retained which then if need be can still be varied within narrow bounds by the attending doctor.

Preferably the components of the system each have a specific codification, so that for example a procedure for the dosage of a medicament allows the activation of a corresponding medication device only.

The signal processing device can be formed in such a way that the medication signal is emitted as a scalar in a range from 0-99.

The spectrometer device can be formed in such a way that it includes a measuring head that can be applied to a selected tissue area of the patient, and that this measuring head over a light guide device is coupled with an analysis module, the signal processing device being integrated in that analysis module.

Further particulars and characteristics of the invention result from the following description in connection with the drawing. The figures show:

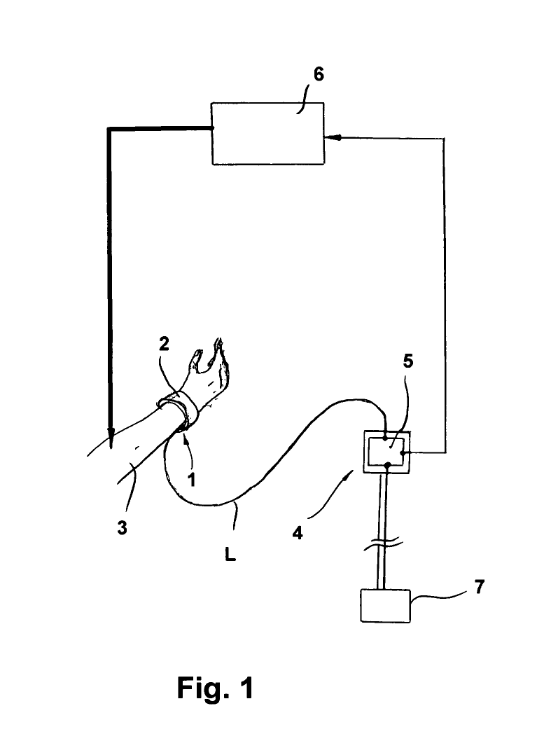

FIG. 1 a sketch to illustrate the structure of a medical equipment system according to the invention.

FIG. 1 shows a medical equipment system according to the invention.

It includes a spectrometer device for collecting measuring signals from living tissue of a patient, especially for the continuous determination of the composition of body liquids as well as of vascular-bound substances which may be only temporarily present in the corresponding tissue area. This spectrometer arrangement includes a measuring head 1, that here typically is connected by a bracelet 2 to a patient 3. This measuring head is connected over a light guide cable L with a measuring device 4. The measuring device 4 comprises the spectrometer device as well as a programmable signal processing device 5 for processing the measuring signal delivered by the spectrometer device. The signal processing device 5 is moreover configured in such a way that by it a medication signal can be generated, by which a medication device 6 is controlled. The medication device 6 serves for the administration of a medicament in accordance with the aforementioned medication signal. The signal processing device presents a first interface for the connection of the spectrometer device, a second interface device for the connection of the medication device, and a third interface device, in which over the third interface device the evaluation of the measuring signals delivered by the spectrometer device and the determination procedure for the medication signal can be configured. After the configuration of the signal processing device a configuration computer 7 can be separated from the system. The medication is done then directly in accordance with the spectrometric measurement on the patient and the program deposited and configured in the signal processing device.

1. A medical equipment system with:
a spectrometer device for collecting measuring signals from living tissue of a patient, especially for the continuous determination of the composition of body liquids as well as of vascular-bound substances which may be only temporarily present in the corresponding tissue area,
a signal processing device for processing the measuring signals delivered by the spectrometer device and for generating a medication signal; and
a medication device for administering a medicament in accordance with the medication signal,
wherein
the signal processing device presents a first interface for the connection of the spectrometer device, a second interface device for the connection of the medication device, and a third interface device, in which over the third interface device the evaluation of the measuring signals delivered by the spectrometer device and the determination procedure for the medication signal can be configured.

2. The equipment system according to claim 1, wherein the spectrometer device and the signal processing device are configured in such a way that by these media the detection of the spectra is done with such a high sampling rate that in this way cell dynamic processes, for example vasomotion effects, can be recognized.

3. The equipment system according to claim 1, wherein by the third interface device signal processing programs can be loaded into the signal processing device, which are kept ready in a computer system as parts of a tool box.

4. The equipment system after claim 1, wherein the medication signal is emitted as a scalar in a range from 0-99.

5. The equipment system after claim 1, wherein the spectrometer device includes a measuring head that can be applied to a selected tissue area of the patient, and that this measuring head over a light guide device is coupled with an analysis module, the signal processing device being integrated in that analysis module.

6. The equipment system after claim 1, wherein the spectrometer device includes a measuring head that can be applied to a selected tissue area of the patient, and that both this measuring head and the signal processing device are integrated in an analysis module attachable to the patient.
